(19) United States
(12) Patent Application Publication
(10) Pub. No.: US 2018/0091640 A1
(43) Pub. Date: Mar. 29, 2018

(54) Cloud based personal services system

(71) Applicants: Paul Fulton, Los Gatos, CA (US); Hans vanLeuen, Amsterdam (NL)

(72) Inventors: Paul Fulton, Los Gatos, CA (US); Hans vanLeuen, Amsterdam (NL)

(21) Appl. No.: 13/896,179

(22) Filed: May 16, 2013

Related U.S. Application Data
(60) Provisional application No. 61/648,010, filed on May 16, 2012.

(51) Int. Cl. H04M 1/725; G06Q 20/32; G07C 9/00; H04W 4/02; G16H 10/60

(52) U.S. Cl. CPC H04M 1/7253; H04M 1/72572; G06Q 20/3278; G06Q 50/01; H04W 4/023; G16H 10/60; G07C 9/00111

(57) ABSTRACT

A personal identification, tracking, entertainment and communication system is provided that is sufficiently flexible and intelligent to address a variety of applications and needs. In one embodiment, an electronic accessory such as an electronic bracelet or the like is provided. The electronic accessory may be inexpensive (disposable), and easily provisioned. When an electronic accessory is issued to a person, personal information identifying that person is stored remotely in association with unique identifier of that electronic accessory. A network of base stations provides communications between such electronic accessories and remote servers. The remote servers provide intelligent applications, analytics capabilities, etc. Various vertical market applications may be provided that are customizable, brandable, etc. Examples of such applications include hospitality, music and entertainment, social networking, corporate guest events, sporting events, public services, disaster relief management, point-of-sale payment, etc. The applications may cooperate with corresponding applications running on mobile electronic devices such as smartphones, tablets, etc.

### The present invention relates to personal services including, for example, personal identification, entertainment, tracking, payment and communications.

### BACKGROUND OF THE INVENTION

Various types of identification bracelets are known, including, for example passive bracelets of a type widely used in hospitals, electronic bracelets or anklets used for monitoring, etc. Also known are novelty entertainment bracelets that may be controlled to light up in a way that is coordinated with being played. None these are sufficiently flexible and intelligent to address a variety of applications and needs.

Examples of passive data collection and data analytics include use of passive radio frequency identifier (RFI) tags, badges to document entry and exit and capturing specific activity at specific monitoring points where passive readers have been placed. Wearable passive systems, however, only provide relevant data on captured information when a passive tag comes into close proximity (range varies by type of tag and reader). Activities that take place between the periods a tag goes from one data collection point to another are not recorded. Data captured by the passive systems also do not allow personalization or active communications back from the end-users. Wide area and local area networking solutions are limited in deployment and use due to issues with high price, size and battery life associated with the personalized user devices. Mobile phone applications also provide a subset of users with a level of functionality and personalization options through Near Field Communications (NFC). NFC provides a secure wireless two-way communication between mobile phones and devices and reception devices such as other mobile phones, payment terminals or identification readers. The smartphone market is expected to adopt NFC as a new connectivity (transaction and personal identification) option. However not all users have access to the mobile devices that support these personalized services and applications. For example, not all young children and senior adults own or carry data service enabled mobile phones. In addition, foreign travelers often do not turn on mobile data due to the high international costs; this is especially pertinent, for example, in large resorts.

### BRIEF DESCRIPTION OF THE DRAWING FIGURES

The present invention may be further understood from the following Detailed Description in conjunction with the appended drawing figures. In the drawing:

FIG. 1 is a diagram of a cloud-based personal services system.

FIG. 2 is a diagram illustrating aspects of personal location services.

FIG. 3 is a diagram illustrating further aspects of personal location services.

FIG. 4 is a diagram of a wireless module that ay be used in the system of FIG. 1.

FIG. 5 is a diagram of a base station that may be used in the system of FIG. 1.

FIG. 6 illustrates an example of a iPad application for disc jockeys (“DJs”) to enhance the enjoyment of music listeners equipped with wristbands or the like.

### DETAILED DESCRIPTION

### Summary

A personal identification, entertainment and communication system is provided that is sufficiently flexible and intelligent to address a variety of applications and needs. In one embodiment, electronic accessory such as an electronic bracelet or the like is provided. The electronic accessory may be inexpensive, disposable, and/or easily provisioned. When this electronic accessory is issued to a person, personal information identifying that person is stored remotely in association with a unique identifier of that electronic accessory. A network of base stations, typically together with one or more gateways, provides communications between such electronic accessories and servers, e.g., application servers. The servers provide intelligent applications, analytics capabilities, etc., and can be remotely located and Internet connected, i.e., “cloud connected.” Various vertical market applications may be provided that are customizable, brandable, etc. Examples of such applications include hospitality, music and entertainment, social networking, disaster relief management, point-of-sale payment, etc. The applications may cooperate with corresponding applications running on mobile electronic devices such as smartphones, tablets, mobile PCs, etc.

### Description

As described herein, opportunities for unique and compelling location based services are created for commercial businesses such as resorts, hospitals, and public service agencies whose customers move throughout their facilities. Aspects of the system include, in one embodiment, the system architecture and component definitions of a wireless radio module, cloud computing, multivariate analysis algorithms, point-to-multi-point and mesh networking protocols, data link protocols, application layer protocols, and mobile applications (e.g., iPhone/Android/iPad applications).

One distinguishing feature of the present system is the concept of personalization through active end-user participation as opposed to passive tracking. Active collection of relevant user behavioral information enables cloud based software applications to interact and influence the end-user's experience based on personal user profile data as well as relevant real-time data that is shared with the application via wearable wireless devices. At least two significant system design elements of the service platform contribute to personalization of the service and application. First, relevant active data are captured by the service, based on variables specific to an application service (end-user activities, location, user transactions, etc.). Second, processing is performed of this relevant active data with other end-user specific information that is stored and accessible via a cloud based application service. The active data that is captured and observed by the service is connected to other relevant databases in the cloud. These connected services form a basis for communications with the end-user. Combining and processing this data and then using the processed results to communicate with the end-user via a low-cost wireless device provides significant benefits, enabling a service that utilizes an active data collection system and is more effective for capturing and analyzing real-time user transactions and behaviors. In one example of an application service, the wireless module is integrated into a wristband to be used to direct large crowds at events, to track family/kids, or to pay for purchases.

The processing of this information may take place in real-time, leveraging the power of cloud computing. Feedback/guidance to the end-user may be communicated via lights, text or sounds via the network connected wearable wireless module. The two-way communications is facilitated by the design of the wireless module, which can be integrated into different enterprise configurable form factors, offering significant advantages over mobile phone and RFID applications and solutions. Commercial enterprises, educational institutions, health care providers, public service organizations and government agencies can utilize this rich data and intuitive user interface and communications channel to develop new and innovative end-user (e.g., patient, student, public service worker, etc.) modeling, training, and marketing programs. These new and innovative personalized user services applications benefit end-users in real time by providing valuable communications and feedback to users requesting assistance or additional services.

With the popularity and robustness of local area networks, mobile wide area networks, active RFID tags, and advances in mesh networking options, it is possible to have deployments of the personalized user services that start small and expand organically to support several thousand concurrent users. Utilizing distinct data collection, wireless, mobile and Internet technologies, a system architecture and application development platform is provided that uses low-cost, low-power, wireless, wearable, devices as the means for collecting and communicating rich active data between the end-users and the supporting commercial enterprise or public service organization. Low cost wireless devices are provided that take advantage of low power silicon and RF solutions, local area network solutions, and wide area networking and mesh networking. Mobile networks, wide area networks, and outdoor and indoor wireless applications may each be used, resulting in a flexible communications architecture. A network based system architecture leverages Internet (cloud) computing, analysis, and storage platforms to enable quick development, large scalability, and high affordability.

The physical area, where the networking system is used, has to be “covered” by base-stations. The usable coverage (or range) per base-station depends on many factors, as antenna type, height, transmitter power output, receiver sensitivity, modulation type, data rate, other RF interferers, environmental physical blockages and absorbers, like trees, buildings and people. These system parameters will be selected and set per application. In addition, the total usable area depends on the number of base-stations and the method of placement. In one example (battery) powered base-stations are to be placed by people without any RF and network knowledge. The base-stations are self-adjusting and/or can be controlled and set-up over internet. A base-station connects to internet with WiFi or Ethernet.

System Architecture and Major Components (Function, Performance, and Interaction)

A system design and architecture provides secure communications between end-users wearing the low cost wireless devices and the wireless network architecture and cloud communications infrastructure required to provide meaningful services to the personalized device users.

The resulting personalized wireless system connects secure low cost personal identification information with location based information to provide meaningful data that is useful to commercial enterprises, while also providing useful and personalized services to the consumers.

In one embodiment, a personal services system may include multiple personal electronic accessories, each including: a one-way or two-way radio; an input device; an output device; and a controller coupled to the two-way radio, the input device and the output device. The system may further include one or more servers for storing personal information associated with respective ones of the wearable electronic accessories; and communications infrastructure including multiple base stations for communicating with the multiple wearable electronic devices and the one or more servers. The system may further include multiple mobile electronic devices configured to communicate with the one or more servers.

In another embodiment, a server or group of servers may include multiple customized applications and be configured to: receive identifying information from a personal electronic accessory; select an application from among the multiple of customized applications and access personal information stored and accessible to the one or more remote servers based at least in part on the identifying information; and run a selected application to provide personal services using the personal information. The server or group of servers may be configured to perform tracking of a large number of personal electronic accessories. The server or group of servers may be configured to perform analytics with respect to tracking information obtained by said tracking.

In some embodiments, the personal information may include payment account information. Methods may further include providing goods or services to a user of the personal electronic accessory; and charging for the goods or services based on identifying information of the personal electronic accessory using the payment account information. The selected application may be customized for an enterprise and the goods or services may be age-limited. Methods may further include sending to the enterprise age verification information based on identifying information of the personal electronic accessory.

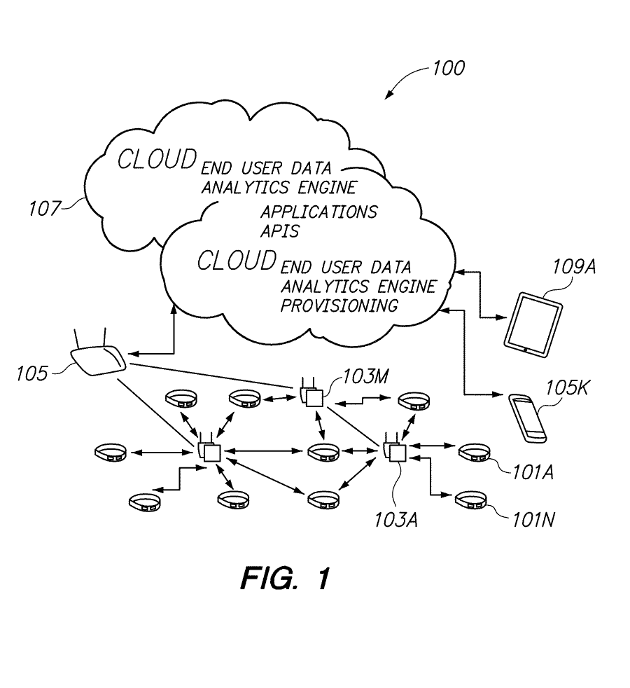

Referring to FIG. 1, a diagram is shown of an exemplary cloud-based personal services system 100. Wireless modules 101A-101N (in this example wristbands) are worn or carried by users and communicate with local base stations 103A-103M. The local base stations communicate in turn with routers 105 such as WiFi, power line or Ethernet Internet routers. The routers route communications to one or more remote servers 107 (“cloud servers”). The cloud servers 107 house end user data, analytics capabilities, applications, and APIs for accessing other cloud or Internet-based services. The cloud servers 107 also handle end user data security and provisioning. The cloud servers 107 communicate with user devices 109A-109K (through the base stations) and with professional Internet connected devices (e.g, iPhones, iPads, smartphones and tablets generally, kiosks, PCs, etc.).

Cloud Services and Mobile Applications

The present service benefits and provides a competitive advantage to government, public service organizations and commercial enterprises, providing them with detailed real-time data that is generated from personalized user sources including relevant connected cloud based social media and enterprise data sources. Having access to this relevant active data and having a direct communications channel back to the end-user enables commercial enterprises and public service organizations an opportunity to act on the data immediately. This results in a valuable communications and research tool for better understanding and mining rich data analytics of user behavior patterns as well as a tool to locate and help people find each other. Current data collection services do not provide easy access to the two-way communications channels, which enables interaction between the public service organization/commercial enterprise and the end-users.

In one embodiment, a method of providing personal services using a personal electronic accessory may include the personal electronic accessory communicating identifying information to one or more remote servers; selecting an application from among a multiple of applications and accessing personal information stored and accessible to the one or more remote servers based at least in part on the identifying information; and running a selected application to provide personal services using the personal information.

Methods may further include tracking locations of multiple personal electronic accessories using a network of base stations, and communicating location information to the one or more remote servers. The selected application may be customized for an enterprise, and the personal electronic accessory may include a button. Methods may further include receiving at the one or more servers an indication that the button of a particular personal electronic accessory has been pressed; and sending a notification to the enterprise that the button of the particular personal electronic accessory has been pressed. Methods may further include sending to the enterprise location information concerning a location of the particular personal electronic accessory. Methods may further include the one or more servers performing analytics using the location information.

In one example, the wireless module is integrated in a wristband to be used to direct large crowd at events, to track family/kids, and to give access to activities, etc. The data collected with the wireless module can be of any kind, such as temperature, unique identifier, battery power level, credits, location, etc. The module makes contact with a local base station at a certain time interval and sends a short message. Upon reception of this message (by the server) an acknowledge message is returned. These “acknowledge” messages may contain information for the user/bearer of the module. The data collected by the wireless module (in the wristband) is transferred to an Internet cloud server through a local wireless base station. Depending on the application, relevant data can be processed at the cloud server. Processing can be “real-time” or at a later moment, when a service requires this (e.g., at request of a user).

In one embodiment, any approved device can access the secure stored data on the cloud server. Approval of access for a certain device can be given to each individual device, or user, on a license basis or per time access basis, for example. Secure access protects data privacy and transfer.

Location and user data is analyzed with cloud computing and processing power. The results can be displayed on a mobile device, tablet or PC device. Examples of suitable mobile electronic devices are Apple's iPhone and iPad mobile devices, Android phones and tablets, Windows phones and tablets, etc. Privacy safeguards may be provided for the data available in the cloud, safeguarding the privacy of the owners of the data and the data itself. The ability to view the cloud-stored data may be made available to smartphone or Internet connected tablet users. (Typically, the application to do this function does not require large memory or processing power.) One function of the application is to set the input for the algorithms running on the server. The information may be displayed graphically on the device.

Examples of commercialized enterprise applications may include:
1. Communications system for logging and recording the daily/weekly monthly activities of visitors or guests to commercial buildings, and campuses.
2. Lost child/guest communications and logging system that facilitates communication between parents, children or other people in a specified group using visual communication methods, including the communication of location information.
3. Health care service monitor or communications connection between patients and the providing health care services organization/hospital/clinic/doctor, including the communication of location information, patient records, etc.
4. Safety and security monitoring service for large commercial enterprises that an to track flow and activities of outside visitors, contractors and employees.
5. Guest services management and communications system for processing and managing flow of hotel guests and visitors (thousands of end-users) at a hotel resort property. Guests could be alerted of marketing programs based on personnel profiles, resort capacity, time of day, etc.
6. Public service notification, access and attendance management system for processing and managing logistical flow of large groups (thousands of end-users) attending one-time events (e.g. sporting, fairs, entertainment parks).

A selected application may be customized for a public venue, and the personal information may include family or friendship relationship information. Methods may further include the one or more remote servers: receiving a report of a lost family member or friend; locating a personal electronic accessory of the lost family member or friend; and sending to the public venue information concerning a location of the personal electronic accessory of the lost family member or friend. Methods may further include displaying on a terminal pictures or video information corresponding to a location of the personal electronic accessory of the lost family member or friend.

Examples of potential government and public service applications may include:
1. Communications system for logging and recording the daily/weekly monthly activities of disabled/disadvantaged users that are dependent on care for their daily/weekly activities.
2. Child/parent communications and logging system that facilitates communication between parents and children using non-verbal visual communication methods.
3. Communications solution for improving end-user flow and monitoring/processing/servicing of end-users (i.e., DMV, Unemployment Office, or other public service agency) for improving overall end-user/client/customer-service processing and end-user requests.
4. Non-first responder, emergency services system for monitoring and providing simple communications for emergency services workers (e.g., American Red Cross).
5. Homeland security, communications platform for monitoring, tracking and communications with end-users in specific designated government and public buildings, or specified areas.
6. Educational system for monitoring student flow, class attendance, and activity/event involvement and participation.
7. Public service notification, access and attendance management system for processing and managing logistical flow of large groups (thousands of end-users) attending one-time events (e.g. speech, public gathering) or visiting a large public service facility.

The cloud based system platform architecture described enables the rapid development of applications and services for commercialization purposes for enterprise applications or for public service organization that are chartered with supporting special user groups, (based on end-user profiles or other variables that are captured and deemed relevant data points by the commercial enterprise or public service organization). Specific innovation and application may include specific user groups that do not have the capability to speak and understand the English language due to physical, mental or cultural variables. The service would determine this based on information in the cloud, e.g. personal user profile, as well as from accessing other relevant information from the wireless module, for example; how long a person stands in front of a directory/sign/electronic ticketing machine, etc.

Networking System Architecture, Communications infrastructure, and Data Analytics

Cloud infrastructure includes data storage and access architecture, analytic software using the cloud services, and interfaces that facilitate easy integration with standard and identified enterprise databases and social networking services. Access to data storage may be through web services interfaces accessible to both enterprise subscribers as well as end-users of the wireless modules and wireless base stations.

A network based architecture design leverages remote analytic processing in the cloud, data storage, communications, interfaces to financial services, educational services, wireless networks (wide area as well as local area networks), legacy enterprise databases, social networks and media and Internet based applications. This network based architecture supports and facilitates development of applications for sharing data, reporting, data analytics, and functional communications between network hosted applications, data bases and the wireless modules that are connected to the overall system architecture via the wireless base stations.

Analytic services analyze end-users behavior using the cloud services and provide feedback to the users.

The system may leverage existing research in the areas of multivariate cloud algorithms and personalized recommender algorithms in distributed information environments in the cloud. Today, personalization is something that occurs separately within each system that one interacts with. Recommender systems are one technique for personalization; in essence the personalization occurs slowly as each system builds up information about end-user likes and dislikes, about what interests the end-user and what fails to interest the end-user. These personalization techniques may rely in part on collection of system usage history which is then employed to change the behavior of the system, or on the user explicitly personalizing the behavior of the system in various ways by setting parameters, making selections or engaging in dialogs with the system.

Wireless Modules and Base Stations (Including Communication Firmware and Protocols)

An important contributing factor to the features and capabilities of the system is a low-cost, miniature, and wearable wireless module. This module provides basic user identification information, as well as location and behavioral information that the user is sharing with the service. The communications protocol and design of the wireless module provides secure two-way communications between the enterprise and the end-user. This two-way communications enables the development of commercial applications where end-user feedback and communications greatly enhances the user experience with the service. The wireless module enables commercialization and branding of specific wearable devices (pendants, wristbands, badges, etc.) that integrate and work with the wireless module. Enterprises and public service organization as well as application developers have access to incorporate the wireless module into different form factors of devices to accommodate specific end-user preferences and applications.

Unlike traditional passive and active RFID systems, where data is batch processed from sources such as passive RFID tags, this service with its associated applications is different because the active data is coming from a user wearable wireless module which transmits relevant information back to the cloud based service that is integrated with other application specific information and application sources, where the end-user can communicate back to the service. This is a significant feature of the service which enables commercial enterprises the opportunity to further commercialize the service by developing and enabling marketing and service assistance programs that are linked to end-user behavior patterns, user communications and logistical geographical flow patterns. For example, an end-user that had a specific profile that indicated they had hearing problems and was in need of assistance could be guided via communications from the application service to the user's wireless module. The service could verify the user received and understood the communications by behavioral actions (movement) of the user. The user could also communicate to the service via the wireless module as well.

A cost effective wireless module and base station reference design provides an effective communications link and communicator to the system architecture. The module design is non obtrusive and has a simple and intuitive user interface and design.

In one embodiment, a personal electronic accessory (or personalized user device) may include: a two-way radio; an input device; an output device; a controller coupled to the two-way radio, the input device and the output device; a housing that houses the two-way radio, the input device, the output device, and the controller; and at least one light pipe and at least one LED, and an LED controller responsive to commands for causing the LED to emit a color of light selected from many possible colors. The housing may include a feature enabling the accessory to be worn by a user; may be waterproof; may, in some embodiments, be in the form of a wrist bracelet.

Depending on system requirements, the design of personalized user device(s), including design specifications of the wireless radio technology and wireless module, design specifications of feature set of wireless module and integration with personalized user devices (e.g. iPhone, Android, tablet), etc., may differ. Personalization options and methodology may also differ.

Suitable wireless modules may have some or all of the following physical specifications:
Wireless module physical design that enables easy integration into different form factors of wearable and portable devices (bracelets, pendants, anklets, key fobs, etc.)
Module holder in silicon rubber to create an end-user wristband
Electronics waterproof
Size ½″ (12.5 mm) wide
Disposable
Non rechargeable
Response time <1 s (glowing when received acknowledgement of the transmitted request)
Small button cell battery with high capacity (may be replaceable)

In one embodiment, the frequency band for wireless module may be either 410 MHz or 915 MHz. A data rate of 20 to 100 kbps with a packet size of 8 up to 64 bytes will typically be sufficient. Any of various different modulations may be used for the wireless module (for example OOK, GFSK and frequency hopping GFSK), together with corresponding communication protocols. An example of a suitable radio chip that may be used by the wireless module of the Silicon Labs Si1000 family, for example the Si1014. In one embodiment, firmware of the wireless module may be updated over the air.

In one embodiment, the system design of the wireless base stations enables support of one-to-many communications with multiple wireless modules. Wireless local base stations are designed to support mains and battery power.

Mesh network have the functional capability to cover large geographic areas, and support multiple thousands of wireless modules. By following a mesh network design, these units are able to simultaneously communicate with multiple other wireless mesh network connected base stations. The mesh network design may be, for example, that of a low-cost, robust, propriety Active Wireless Mesh Network (AWMN). Such a mesh network is particularly suitable for areas where the local access points do not have access to WiFi, power line or Ethernet network communications, or where access would render the product too expensive.

Typically, using RF transceivers for data communication requires knowledge about RF behavior. Mesh-networking with self-installing properties, on the other hand, allows for easy installations by enterprise customers without RF knowledge. The mesh networking protocol may based on propriety RF or on WiFi, using standard chips and modulation technologies. In one embodiment, the protocol is optimized for low data rate (small) packets, and supports network nodes that can communicate which each other and transmit a message generated by one unit to other physical locations where an Internet (or other) connection is available. By having all local base station devices mesh network with multiple devices, the range and coverage of the network may be extended with or without Internet connectivity. The network architecture and wireless protocol algorithm enable this mesh networking to take place. The mesh network may use any of various suitable protocols, including WiFi, proprietary RF protocols, etc.

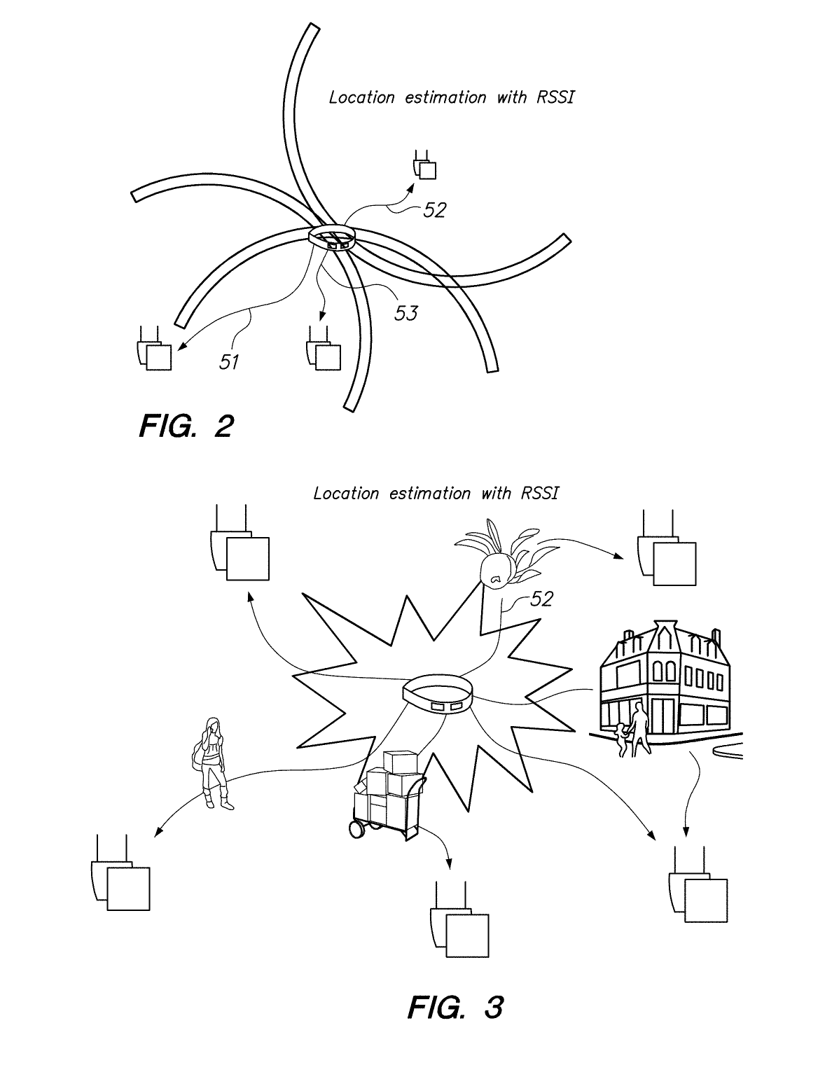

Referring to FIG. 2, in one embodiment, location identification is performed by measuring signal strength. Location identification by measuring signal strength is not as accurate as GPS, but is significantly cheaper in power and cost. In contrast to GPS, this solution can work indoors. It should be noted that an indication of the received signal strength (RSSI) varies highly with RF link conditions, such as wet/dry environment, reflections off of people, furniture or trees, etc. Therefore, multiple receivers at relatively small distances, up to 100 m for instance, are required to determine a trusted and accurate position. In the described use models, the accuracy need not be very high. An accuracy range of 10 m will often be sufficient, and positioning the base stations 30 to 300 m apart will work for most applications. The required connection between the receivers (local base stations) can be realized through RF mesh, Ethernet or WiFi networking. As indicated above, the distance to a base station can be estimated by received signal strength. There is a relation between the signal strength and the distance between transmitter and receiver; by measuring the received signal over a certain time interval and possibly combining with measured data from multiple base stations, a location can be calculated.

Each wireless module transmitter will be at a certain distance from the receiver (the base station). This distance is a circle (or sphere) around the base station. In FIG. 2, this is indicated by the orange curves. The intersections of the orange curves, of which the signal strength S1 . . . S3 are the radius, is indicating the area where the wireless module resides. Because of practical natural influences, like reflections of the RF attenuation, absoption, etc., the signal paths are not exact in relation to the signal strengths. This is depicted in FIG. 3, showing that the location cannot exactly be determined. The accuracy of the estimated location varies therefore in an unpredictable manner. Using more base stations increases the location accuracy. Calculations may be performed on the remote cloud or local servers to reduce hardware cost and battery power consumption at the local devices.

A location operation of a wireless module may be performed in accordance with the following steps:
Wireless module message is received by multiple base stations.
Received Signal Strength Indication (RSSI), is measured, the location is determined by a cloud server and the data message is processed according to application.
The location is sent to companion device (e.g. smartphone or tablet).
The location accuracy can be adjusted, depending on the use case requirements.
Service provider gets service call and contacts the end-user.
Companion device uses an application to track location and display the relevant data.

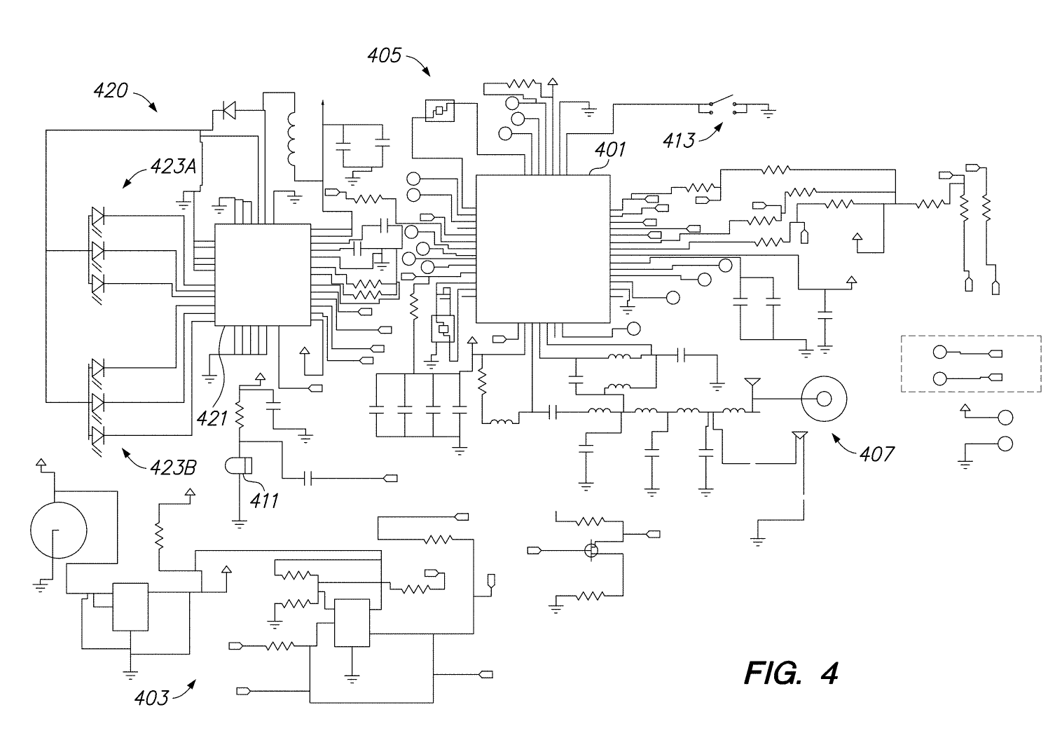

Referring to FIG. 4, a diagram is shown of a wireless module in accordance with an exemplary embodiment. An RF microcontroller 401 is coupled to a power section 403, a clock section 405, an RF path 407, an input section, and an output section 420. The RF path 407 enables the wireless module to communicate with base stations. The input section may include a microphone 411 (e.g., a MEMS microphone) and one or more input switches 413. In other embodiments, any of various other input devices may be used. The output section 420 may include an LED driver 421 and one or more LEDs 423 (in the illustrated embodiment, two LEDs 423A and 423B, one for the top side of a wristband and one for the bottom side of a wristband). The LEDs may be color (e.g., RGB) LEDs. In other embodiments, any of various other output devices may be used, including for example a display (e.g., a 20-character display, a graphic display, etc.), a speaker, a vibrator, etc.

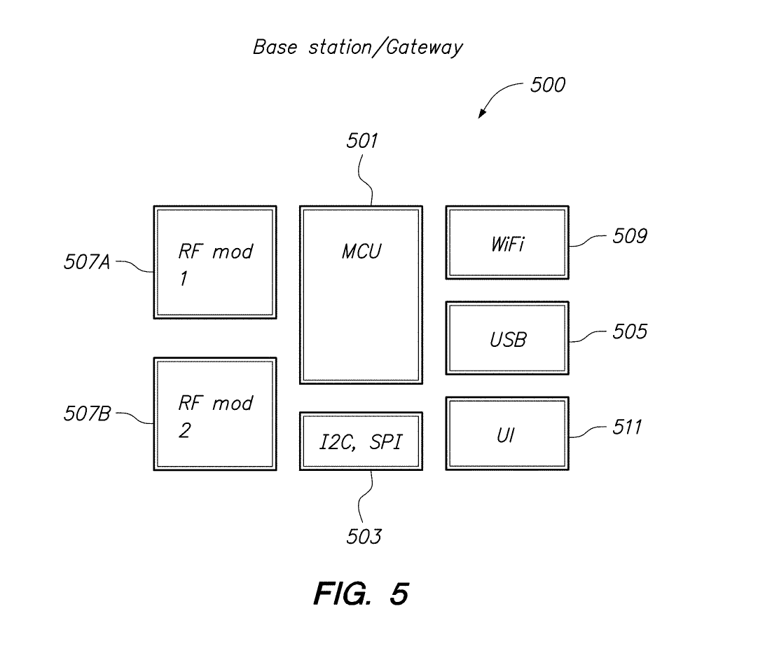

Referring to FIG. 5, a diagram is shown of a base station in accordance with an exemplary embodiment. A microcontroller unit (“microcontroller”) 501 is coupled to wired connectivity circuitry including, for example, internal connectivity circuitry 503 such as I²C and SPI, and external connectivity circuitry 505 such as USB. The microcontroller 501 is also coupled to wireless connectivity circuitry 507. One or more RF modules are used to communicate with wireless modules worn or carried by users. In the illustrated example, two RF modules 507A, 507B are provided, to provide additional capacity and allow for increased location capabilities. A WiFi module 509 may be provided to communicate via wireless and wired infrastructure to one or more servers that provide cloud-based personal services. In other embodiments, the base station may communicate to the one or more servers through further communications equipment interfaced through the USB port, for example. User interface circuitry 511 is provided to allow for user interface and interaction with the base stations, by employee-users, end-users, or both. In one embodiment, the microcontroller is an ARMS microcontroller and runs the Linux operating system or a variant thereof.

End-User Feedback Analysis

The present system provides a valuable research tool for better understanding and mining the rich data analytics of user behavior patterns and in determining variables that affect user trends and logistical flow patterns.

End-user feedback analysis may be performed to take account of experience with the device in terms of, for example, form factor, rechargability vs. disposability, etc. Of particular interest is the behavioral effects of providing a communications link to end-users/customers and to see what impact this has on the communications paradigm, which shifts from a from a passive, “I'm being tracked,” paradigm to an active paradigm that says, “I'm participating and my individual activities and behavior patterns are analyzed and help influence and shape my overall experience”. As a result, earing the device is expected to impact user behavior in the location area(s). Various trials may be performed to determine what information and feedback end-users would like to have communicated back to them. Trials may also be performed to determine the desired methodology for communications with end-users (e.g. lights, sounds, text, etc.).

Software As A Service (SAAS)

The foregoing location based services are instantiated in cloud and mobile applications may be offered in accordance with a SAAS business model, sold directly to initial “lighthouse” customers, but also expanding to sales through vertical channel partners later in the product adoption cycle. In addition, base station products, wireless modules, wireless module holders (wristbands, pendants, etc.) and installation services may be sold as products or services respectively. The flexibility of the underlying platform allows for third party companies to add services, applications, or alternative/custom wireless module holders.

Additional Aspects

Additional aspects of the present cloud-based personal services system include the following:

System for remote personal paging, people and item tracking, finding parked cars, purchases, security requests, help requests, access control for resorts (including resorts on cruise ships), hospitals, clubs, emergency relief centers, sporting events (golf, NFL, NHL, MLB, Nascar, Soccer, etc.), concerts (music, DJ, etc.), conferences, conventions, whereby patrons wearing device can summon staff at event by pressing button on wearable wireless module (built into wristband, bracelet, pendant, badge, necklace, etc). Wireless module sends message to base stations. Base stations send message to Internet. Devices on premise such as iPhones, iPads, android phones, computers, then notify staff of needed service. Notifications include patrons location, time waiting service, patrons information that was stored in the “cloud”.

Wireless module being capable of being installed in many instantiations of wearable products such as wristband, bracelet, pendant, badge, necklace, etc.

System correlates information that is stored in our system in the “cloud”. Wireless modules have unique identifier whereas each patron wearing wireless module can access their information securely and safely. Wireless modules can be used for patron access to facilities, events, and privileges using unique identifier and location for secure access.

Methods may further include granting users of personal electronic accessories access to premises based on identifying information of the personal electronic accessories. For example, the personal electronic accessory may include a proximity communication link and access may be unattended. Methods may further include a reader reading the identifying information from a personal electronic accessory and sending the identifying information to the one or ore remote servers; the one or more remote servers sending a command to an access control device allowing access if authorized according to personal information corresponding to the identifying information.

Wireless modules provide real time location information to cloud that allows for tracking of movement of all wearers of modules at all times.

The module and wireless protocol transfers a minimum amount of data with a minimum of battery power to the cloud through the base station, so a client can get an as accurate possible location.

Wireless modules provide NFC for purchases.

Wireless modules may provide indications of alerts via LED flashing, LED color, voice, voice messages, vibration, small electrical shock, etc.

More particularly, in relation to voice communications, in one embodiment, a selected application may be customized for an enterprise and the personal electronic accessory may include a microphone. Methods may further include the one or more remote servers receiving a voice communication from a personal electronic accessory and relaying the voice communication to the enterprise.

A selected application may be customized for an enterprise and the personal electronic accessory may include a speaker. Methods may further include the one or more remote servers receiving a voice communication from the enterprise and relaying the voice communication to a personal electronic accessory.

The personal electronic accessory may include at least one LED and at least one button. Methods may further include alerting a user of a personal electronic accessory of a voice message using an LED; and relaying the voice communication to the personal electronic accessory in response to activation of a button.

The personal information may include information defining a communications group and the personal electronic accessory may include a microphone and a speaker. Methods may further include: the one or more remote servers receiving a voice communication from a personal electronic accessory belonging to a communication group; and the one or more remote servers relaying the voice communication to other personal electronic devices belonging to the communications group.

Wireless modules provide LED lighting such that LEDs can be controlled in flash rate (on ramp, dwell on, off ramp, dwell off), color, and intensity. System allows LED lighting to be controlled via industry standard DMX lighting commands/controls.

System provides for show lighting where leader (DJ, band member, MC, etc.) can use RF “wand” that allows for a narrow beam of RF power to be shown onto the crowd in a waving motion. When the wand moves over the crowd, the affected wireless modules enact a programmable action such as changing the color, flash rate, on/off, or intensity of the LED's in such a way as to make the people wearing the bands “stand out” and to make patterns in the crowd via waving the wand.

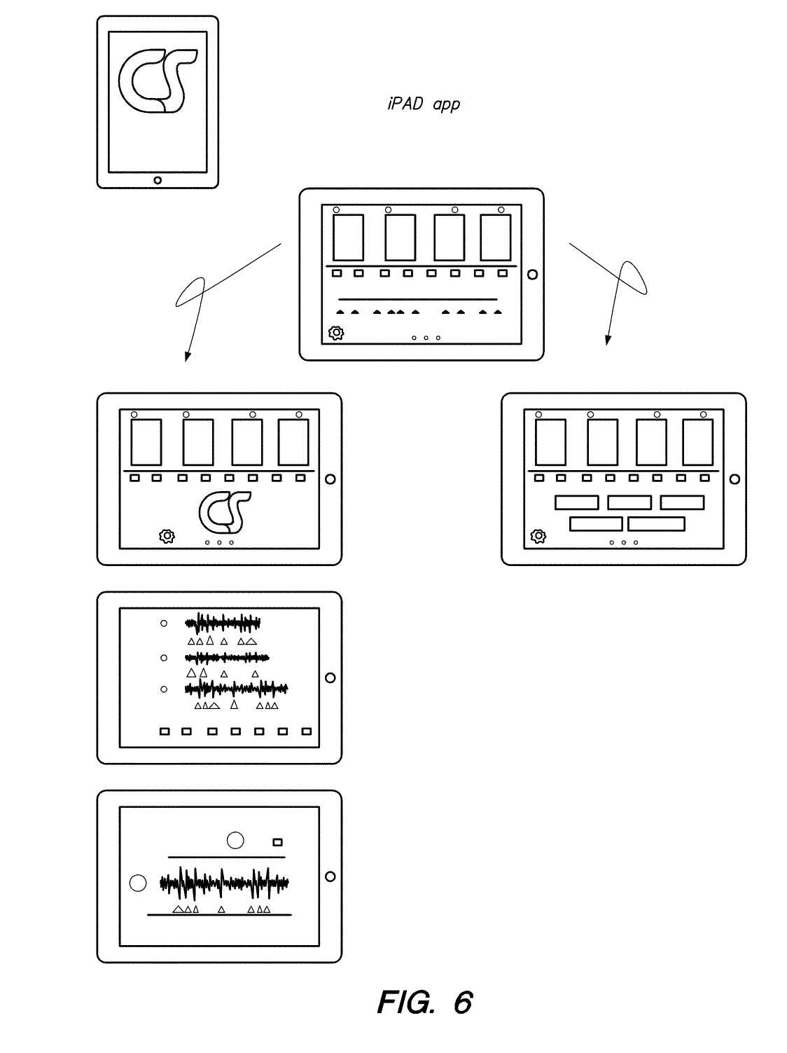

In other embodiments, a DJ may use a iPad or tablet application to cause the bands of people one or more selected areas to light up, vibrate, etc., in a prescribed fashion, or even a random fashion. Such an application is illustrated in FIG. 6.

Wireless modules have capability to “listen” to music with a microphone to allow the modules to “flash” to the beat of the music with or without a control system (stand alone our side the reach of base stations controlled area). Modules can be programmed to “flash” in a variety of ways and respond to audio amplitude or frequency. Depending on the detected power in a certain audio level or frequency band a Red, Green or Blue LED lights up.

Wireless modules can be controlled (on ramp, dwell on, off ramp, dwell off), color, and intensity) via iPhone, iPad, smartphone, computer, Internet by and for individuals and groups for the purposes of:
a. Entertainment—control the flash, color, and intensity of the light
b. Social gaming—play games with voting, proximity, likes matching
c. Dating—matching, proximity, likes matching, flirting
d. Matching interests—coordinate online likes with real life interaction
e. Contests—voting, liking, participating
f. Automatic check-ins on Facebook, Foursquare
g. Identifying and finding lost children, adults, vehicles, items, etc.

In one embodiment, the personal electronic accessory may include at least one light pipe and at least one LED. Methods may further include the one or more remote servers receiving a lighting command; and the one or more servers sending second responsive lighting commands to one or more personal electronic accessories through a network of base stations. The lighting command may be a DMX lighting command. Methods may further include the one or more remote servers selecting a subset of personal electronic accessories to which to send the responsive lighting commands based on the personal information.

Wireless modules can be controlled by user via voice commands, speaking into wireless module or into iPhone, iPad, computer, other interact connected devices etc.

Services: Timing, recording, and analyzing the time it takes between service requests (button pressing) and service arrival time (waitress/server/service provider turns off bracelet when arriving at guest/patient/client).

The system may store multiple “personas” in the cloud. In other words, for each person registered on the system, the system can store information about them that differs based upon their location. Person A may store certain credit cards, likes, and behaviors for a resort, but have different credit cards, likes, and behaviors while at work or at a different resort.

Hence, in one embodiment, the one or more remote servers may be configured to allow multiple distinct personas to be defined for a single person, include storing the personal information so as to allow it to be retrieved by persona.

The system may store patient's health care records in the cloud accessible based upon the wireless module unique identifier.

Hence, in one embodiment, a selected application may be customized for a health care enterprise. Methods may further include sending to the health care enterprise medical records information based on identifying information of the personal electronic accessory.

The system can inform Public Safety of the location and user identification of a user when they press the button on the wireless module.

The system can inform heath care workers of the location and user identification of a user when they press the button on the wireless module.

The system can inform services personnel of the location and user identification of a user when they press the button on the wireless module.

In one embodiment, the selected application may be customized for incident management. Methods may further include the one or more remote servers receiving a request for assistance responsive to a user pushing the button of a personal electronic accessory; and the one or more remote servers sending a notification to incident management personnel of the request for assistance.

Furthermore, the personal electronic accessory may include a lighted indicator. Methods may further include the one or more servers receiving from incident management personnel information identifying one or more personal electronic accessories; and the one or more servers sending commands to the one or more personal electronic accessories causing the lighted indicators of the one or more personal electronic accessories to be lit, signaling to users of the personal electronic accessories to report to one or more locations. Methods may further include the one or more remote servers tracking locations of the personal electronic accessories following signaling to users of the personal electronic accessories to report to one or more locations, and the one or more remote servers controlling the lighted indicators of the one or more personal electronic accessories to vary in accordance with increasing or decreasing proximity to the one or more locations.

The system can provide the location and user identification of a user to his or her Facebook profile page when they press the button on the wireless module.

The system can provide the location on Google maps and user identification of a user to his or her Facebook profile page when they press the button on the wireless module.

The system can provide the video feed of the resorts or events (e.g., foot race, concert, ski resort, theme park, golf resort, etc.) of a user to his or her Facebook profile page, or friends mobile phone, care givers mobile phone, etc. when they press the button on the wireless module.

Hence, in one embodiment, methods may further include the one or more remote servers interfacing with a social networking site to provide at least one of the following types of information concerning a user of a personal electronic accessory to the social networking site: location information; photographic information; videographic information. The selected application may be customized for an enterprise and the type of information may be photographic information or videographic information, and the photographic information or videographic information may be captured by the enterprise and identified to the one or more remote servers. The one or more remote servers may then perform at least one of: storing the photographic or videographic information in correspondence to identifying information of one or more personal electronic accessories; linking to the photographic or videographic information in correspondence to identifying information of one or more personal electronic accessories; and sending or setting up an outside link to the photographic or videographic information.

The system can provide a resort (e.g., ski resort, theme park, golf resort, etc.) management of a guest's location. The system can then correlate the guest's location with revenue factors per location that would then allow the resort to entice the guest to move to different locations to maximize the resorts revenue. For example, the system could communicate to every guest that spends too much time in their rooms, offering them specials to get them out to restaurants, bars, or excursions. In another example, the wearable device with the integrated wireless module lights up or vibrates when the user is in close proximity to an area that the resort is featuring.

The system can provide wait staff with the proper identification of guests when they make a purchase with the wireless module. For example, the guest's wireless module is read and a table computer (e.g., iPad) shows the users Drivers License or passport, VIP status, room number, and credit allowance.

Wireless module holders. The wireless modules are designed such that they can be placed, by the end user or as a service, in a number of holders that can be purchased separately for additional revenue. The holders can include: wristbands, pendants, necklaces, belts, batons, balloons, stick on wall or ceiling modules, undercarriage of car modules, hats, headbands, shirts, shorts, bikinis, hairpieces, backpacks, eyeglasses, drink glasses, rings, watches, pets, cars, bicycles, skis, shoes, socks, etc.

The system allows for the following wireless lighting controls: colors, light intensity, flashing (ramp up time, dwell on time, ramp down time, dwell off time, flashing to the beat or tempo of music, flashing to speaking, flashing to instruments (e.g., cymbals, bass drum, lead guitar, rhythm guitar, lead violin, rhythm violin, etc.), flashing to sound frequencies, location based lighting such as: moving color, intensity, or flash pattern from side of room to side of room, from center of room to edges of room, in wave patterns, etc., person based lighting such as moving color, intensity, or flash pattern based upon characteristics of people such as, male, female, straight, gay, single, rich, musician, dancer, and any other type of personality trait.

The system can communicate with a phone (or PC, or other Device) using audio or light. The microphone can detect audio signals generated by the loudspeaker of the device, while the video of the device can detect the LED of the module. An app running on the phone will be able to communicate with the wristband for instance to transfer the wristband ID to secure log in on the CloudSona website or to set certain parameters in the wristband.

The system can define a certain basestation to radiate in a limited area with a directive antenna and so define a certain function in a certain area. The wristband will for instance light up a certain color, or the ID can be transmitted etc.

The wristband can contain an inductor coil to receive energy to initiate certain functions, like transmitting the wristband ID, or it can be used to charge the wristband battery.

The wristband can transmit a very small packet to indicate it is alive, which in turn can be used for location awareness, or activating other functions.

A single or dual battery can be used in the wristband without changing the circuitry so certain applications with more or less energy requirements can be using one or two batteries.

A receiver with directive antenna can be used to detect the availability of a certain wristband in a certain area.

A receiver with a directive antenna can be used to wake up the wristband. The wrist band can detect the transmitted signal pattern and decodes the sequence. The RF signal is received by a sensitive receiver, which rectifies the power, which in turn is fed through a digital PN correlator, which correlates the signal with a pre set code, which then, when the code is correct, is amplified and throws a switch which wakes up the module.

The communication protocol is developed such that:
Packets are short
Module Response time 20 ms-10 s depending on settings
ACK or NACK on or off
Flash Pattern is stored in the module, it is selected by a message (1 out of 8)
Flash pattern can be reprogrammed wirelessly from iPad
Flash pattern can be defined on the iPad and then send to a certain group
First filter is one byte and filter does not take longer than that. The system can go power down immediately after the filter result is negative

Antenna module is in the frame

It will be apparent to those of ordinary skill in the art that the present invention can be embodied in other specific forms without departing from the spirit or essential character thereof The foregoing description is therefore to be regarded as illustrative, not restrictive. The scope of the invention is defined by the appended claims, not the foregoing description, and all changes which some within the range of scope of equivalents thereof are intended to be embraced therein.

1. A method of providing personal services using a personal electronic accessory, comprising:
the personal electronic accessory communicating identifying information to one or more remote servers;
based on the identifying information, selecting an application from among a plurality of applications and accessing personal information stored and accessible to the one or more remote servers; and
running a selected application to provide personal services using the personal information.

2. The method of claim 1, comprising tracking locations of a plurality of personal electronic accessories using a network of base stations, and communicating location information to the one or more remote servers.

3. The method of claim 2, wherein the selected application is customized for an enterprise, and wherein the personal electronic accessory comprises a button, further comprising:
receiving at the one or more servers an indication that the button of a particular personal electronic accessory has been pressed; and
sending a notification to the enterprise that the button of the particular personal electronic accessory has been pressed.

4. The method of claim 3, further comprising sending to the enterprise location information concerning a location of the particular personal electronic accessory.

5. The method of claim 2, comprising the one or more servers performing analytics using the location information.

6. The method of claim 1, wherein the one or more remote servers are configured to allow multiple distinct personas to be defined for a single person, further comprising storing the personal information so as to allow it to be retrieved by persona.

7. The method of claim 1, wherein the personal information includes payment account information.

8. The method of claim 7, comprising:
providing goods or services to a user of the personal electronic accessory; and
charging for the goods or services based on a identifying information of the personal electronic accessory using the payment account information.

9. The method of claim 8, wherein the selected application is customized for an enterprise and wherein the goods or services are age-limited, further comprising sending to the enterprise age verification information based on identifying information of the personal electronic accessory.

10. The method of claim 1, wherein the selected application is customized for a health care enterprise, further comprising sending to the health care enterprise medical records information based on identifying information of the personal electronic accessory.

11. The method of claim 1, wherein the personal electronic accessory comprises at least one light pipe and at least one LED, further comprising:
the one or more remote servers receiving a lighting command; and
the one or more servers second responsive lighting commands to a plurality of personal electronic accessories through a network of base stations.

12. The method of claim 11, wherein the lighting command is a DMX lighting command.

13. The method of claim 11, further comprising the one or more remote servers selecting a subset of personal electronic accessories to which to send the responsive lighting commands based on the personal information.

14. The method of claim 2, comprising the one or more remote servers interfacing with a social networking site to provide at least one of the following types of information concerning a user of a personal electronic accessory to the social networking site: location information; photographic information; videographic information.

15. The method of claim 14, wherein the selected application is customized for an enterprise and the type of information is photographic information or videographic information, and wherein the photographic information or videographic information is captured by the enterprise and identified to the one or more remote servers, wherein the one or more remote servers perform at least one of: storing the photographic or videographic information in correspondence to identifying information of one or more personal electronic accessories; linking to the photographic or videographic information in correspondence to identifying information of one or more personal electronic accessories; and sending or setting up an outside link to the photographic or videographic information.

16. The method of claim 4, wherein the selected application is customized for incident management, further comprising:
the one or more remote servers receiving a request for assistance responsive to a user pushing the button of a personal electronic accessory; and
the one or more remote servers sending a notification to incident management personnel of the request for assistance.

17. The method of claim 16, wherein the personal electronic accessory comprises a lighted indicator and wherein the selected application is customized for incident management, further comprising:
the one or more servers receiving from incident management personnel information identifying one or more personal electronic accessories; and
the one or more servers sending commands to the one or more personal electronic accessories causing the lighted indicators of the one or more personal electronic accessories to be lit, signalling to users of the personal electronic accessories to report to one or more locations.

18. The method of claim 16, further comprising the one or more remote servers tracking locations of the personal electronic accessories following signalling to users of the personal electronic accessories to report to one or more locations, and the one or more remote servers controlling the lighted indicators of the one or more personal electronic accessories to vary in accordance with increasing or decreasing proximity to the one or more locations.

19. The method of claim 2, further comprising granting users of personal electronic accessories access to premises based on identifying information of the personal electronic accessories.

20. The method of claim 19, wherein the personal electronic accessory comprises a proximity communication link and access is unattended, further comprising:
a reader reading the identifying information from a personal electronic accessory and sending the identifying information to the one or more remote servers;
the one or more remote servers sending a command to an access control device allowing access if authorized according to personal information corresponding to the identifying information.

21.-36. (canceled)
